(19) United States
(12) Patent Application Publication
(10) Pub. No.: US 2019/0329075 A1
(43) Pub. Date: Oct. 31, 2019

(54) ULTRASONIC SONOTHROMBOLYSIS TREATMENT PLANNING

(71) Applicant: KONINKLIJKE PHILIPS N.V., EINDHOVEN (NL)

(72) Inventors: JONATHAN THOMAS SUTTON, BOSTON, MA (US); RALF SEIP, CARMEL, NY (US); WILLIAM TAO SHI, WAKEFIELD, MA (US); JEFFRY EARL POWERS, BAINBRIDGE ISLAND, MA (US)

(21) Appl. No.: 16/466,407

(22) PCT Filed: Dec. 6, 2017
(86) PCT No.: PCT/EP2017/081594

§ 371 Date: Jun. 4, 2019

Related U.S. Application Data
(60) Provisional application No. 62/430,963, filed on Dec. 7, 2016.

(51) Int. Cl. A61N 7/00; A61B 8/14; A61B 8/06; A61B 8/08; A61B 5/00; A61B 5/055; A61B 6/03; A61B 6/00; A61B 34/10

(52) U.S. Cl. CPC A61N 7/00; A61B 8/145; A61B 8/06; A61B 8/0891; A61B 5/0036; A61B 5/055; A61B 8/4227; A61B 6/504; A61B 34/10; A61B 8/0808; A61N 2007/0052; A61N 2007/0078; A61N 2007/0095; A61B 6/032

(57) ABSTRACT

An ultrasound system utilizes an array transducer to perform sonothrombolysis treatment. The system also produces a vascular map of flow characteristics in the vicinity of the therapy site in a subject. The vascular map is used to formulate a treatment plan which includes the number, focusing, timing, and steering of beams of a pattern of therapy beams transmitted during a transmission interval. Formulation of the treatment plan considers factors such as the direction of microbubble flow toward a therapy site, the flow velocity, the spacing between successive therapy beam transmissions, the number of therapy beams needed to “paint” a therapy region, and grating lobe locations, many of which can be determined from the vascular map.

### RELATED APPLICATION

This application claims the benefit of and priority to U.S. 62/430,963, filed Dec. 7, 2016, which is incorporated by reference in its entirety.

### TECHNICAL FIELD

This invention relates to medical ultrasound systems and, in particular, to ultrasound systems which perform imaging and therapy by sonothrombolysis.

### BACKGROUND

Ischemic stroke is one of the most debilitating disorders known to medicine. The blockage of the flow of blood to the brain can rapidly result in paralysis or death. Attempts to achieve recanalization through thrombolytic drug therapy such as treatment with tissue plasminogen activator (tPA) has been reported to cause symptomatic intracerebral hemorrhage in a number of cases. Advances in the diagnosis and treatment of this crippling affliction are the subject of continuing medical research.

U.S. Pat. No. 8,211,023 (Swan et al.) describes an ultrasound system which provides microbubble-mediated therapy to a thrombus such as one causing ischemic stroke, a procedure referred to as sonothrombolysis. Microbubbles are infused, delivered in a bolus injection, or developed in the bloodstream and flow to the vicinity of a thrombus. Ultrasound energy is delivered to the microbubbles at the site of the thrombus to disrupt or rupture the microbubbles. This energetic microbubble activity can in many instances aid in dissolving or breaking up the blood clot and returning a nourishing flow of blood to the brain and other organs. Such microbubble activity can be used to deliver drugs encapsulated in microbubble shells, and well as microbubble-mediated sonothrombolysis.

The Swan et al. patent shows the ultrasonic energy being delivered for sonothrombolysis by an ultrasound beam aimed at a blood clot from an ultrasound array probe controlled by an ultrasound system, e.g. via a single beam in a signal direction.

### SUMMARY

The present invention recognizes sonothrombolysis therapies (and other therapies utilizing ultrasound disruption of microbubbles or other vascular resonators) that involve merely targeting the treatment locale are often inefficient due to premature oscillation and destruction of microbubbles or other vascular resonators near or at the treatment site. For example, grating lobes from the ultrasound beam can cause enough pressure to lyse the microbubbles and reduce their quantity to a level which is not effective to disrupt a vessel occlusion. This often leads to longer treatment times (requiring multiple applications of microbubble delivery and sonication) and/or ineffective therapy.

The present invention solves these problem with an automated treatment plan that tailors timing and positioning of therapeutic ultrasound beams based on several vasculature parameters (e.g., vessel locale, locale of treatment site or clot in the vessel, blood flow direction) and vascular resonator disruption. This enables focused application of acoustic beams to a treatment zone at a time and position when microbubble disruption would be most effective. This invention enables a desired amount of acoustic therapy to be applied to a specific tissue target, in the presence of channels of flowing blood containing vascular resonators.

It is an object of the present invention to plan a sonothrombolysis procedure, including the location and timing of the beam pattern delivering the ultrasonic energy, in consideration of the physiological makeup of a thrombus and the physiology of the subject.

It is a further object of the present invention to plan a sonothrombolysis procedure from an understanding of the vasculature surrounding a thrombus and which delivers the flow of vascular resonators (e.g., gas-filled microbubbles, drug-filled microbubbles, phase-shift emulsions and polymeric cups) necessary for the procedure to the treatment site. While the description often refers to disruption of microbubbles, it is understood that the objects and principles described herein can be applied to any vascular resonator.

In accordance with the principles of the present invention, an ultrasound system is described which performs therapeutic ultrasound treatment using diagnostic imaging to generate a vascular map of the vasculature delivering a flow of vascular resonators to the site of a procedure. The vascular map reveals the topography of resonator flow, which guides the planning of the targeting of the therapy beam pattern in both timing and location. The resulting therapy plan is formulated in consideration of factors such as the direction of resonator flow toward a therapy site, the flow velocity, the spacing between successive therapy beam transmissions, the number of therapy beams needed to “paint” a therapy region, and grating lobe locations, many of which can be determined from the vascular map. The treatment procedure then proceeds in accordance with the planned therapy beam control which is executed by the system transmit controller, subject to updating as dictated by the progress of the procedure.

While the detailed description below specifies use of vascular mapping for sonothrombolysis ultrasound therapy, it is understood that the present invention enables focused application of acoustic therapy for targeted vascular resonator disruption that yields improved and efficient therapeutic results in a wide variety of therapeutic treatments. As such, the present invention is applicable to any application that relies on sonification of microbubbles or other vascular resonators in the vasculature to yield a therapeutic effect. For example, systems and methods described herein would also be relevant to technologies that use vascular resonators to elicit blood brain barrier disruption, sensitization of tissues to drug delivery (e.g. chemotherapy, stem cells, nanotherapeutics, antibodies, viruses, nuclear material, biomolecules, vasoactive compounds), or tissue ablation.

### BRIEF DESCRIPTION OF DRAWINGS

In the drawings:

FIG. 1 illustrates in block diagram form an ultrasonic diagnostic imaging and therapy system for ultrasonic transcranial therapy planning in accordance with the principles of the present invention.

FIG. 2 illustrate the delivery of sonothrombolysis therapy in a two-dimensional (2D) imaging plane

FIG. 3 illustrates the delivery of sonothrombolysis therapy in a three-dimensional image volume which may be imaged for vascular mapping in accordance with the present invention.

FIG. 4 illustrates a probe and headset for sonothrombolysis therapy modeled on the head of a mannequin.

FIG. 5 illustrates the presence of ultrasonic energy in the cranium during beam delivery to a number of target locations in the brain.

FIGS. 6a and 6b illustrate two plans for sonothrombolysis beam delivery planned in consideration of direction of flow of microbubbles to a treatment site.

FIGS. 7a and 7b illustrate two plans for sonothrombolysis beam delivery planned in consideration of spatial and temporal beam separation.

FIG. 8 illustrates a default pattern of sonothrombolysis beam delivery prior to sonothrombolysis treatment planning.

FIG. 9 illustrates a pattern of sonothrombolysis beam delivery formulated by treatment planning in accordance with the present invention.

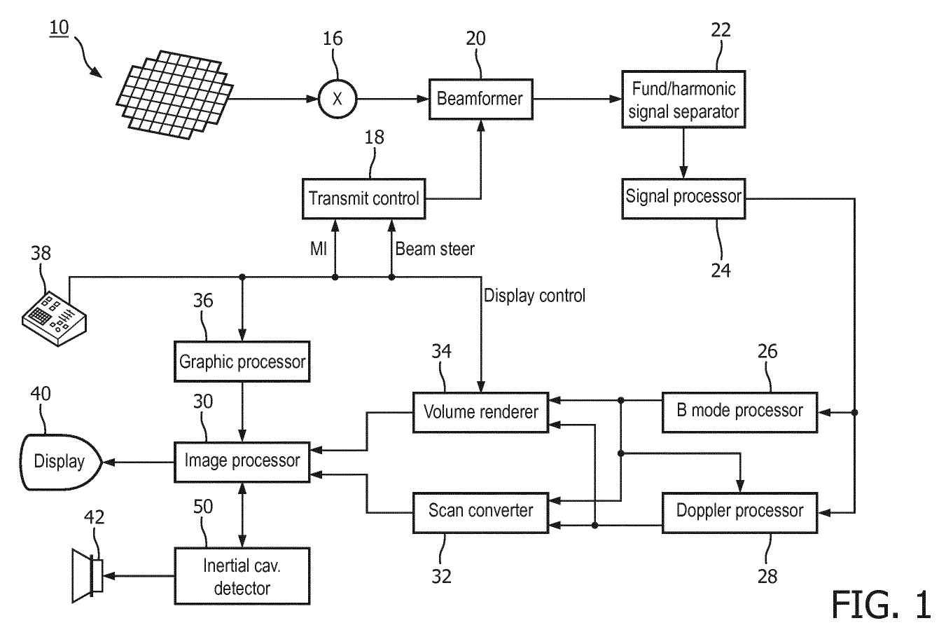

Referring to FIG. 1, an ultrasound system constructed in accordance with the principles of the present invention is shown in block diagram form. A two-dimensional transducer array 10 is provided for transmitting ultrasonic waves for therapy and other uses as described below and receiving echo information. In this implementation, the array is a two-dimensional array of transducer elements capable of steering therapeutic waves in three dimensions and providing 3D images and other information. The array is located in an ultrasound probe which mounts on a headset that locates the array in acoustic contact with the temple on the side of the head for transcranial delivery of sonothrombolysis. In the system of FIG. 1, the transducer array used to deliver therapeutic waves or beams also receives echo signals for image formation and vascular mapping as described below, but in an implementation in which a vascular map is provided by a different ultrasound system or probe or other diagnostic imaging modality, the reception of echo signals by the therapy array and probe is not necessary. The elements of the array are coupled to a transmit/receive (T/R) switch 16 which switches between transmission and reception and protects the receive channels of the system beamformer 20 from high energy transmit signals. The transmission of ultrasonic pulses from the transducer array 10 is directed by the transmit controller 18 coupled to the beamformer 20, which receives input from the user's operation of the user interface or control panel 38, including a planned sequence of beam transmission for therapy.

The echo signals received by elements of the array 10 are coupled to the system beamformer 20 where the signals are combined into coherent beamformed signals. For example, the system beamformer 20 in this example has 128 channels, each of which drives an element of the array to transmit energy for therapy or imaging, and receives echo signals from one of the transducer elements. In this way, the array is controlled to transmit steered beams of energy aimed and focused at specific target locations in the body and to steer and focus received beams of echo signals.

The beamformed receive signals are coupled to a fundamental/harmonic signal separator 22. The separator 22 acts to separate linear and nonlinear signals so as to enable the identification of the strongly nonlinear echo signals returned from microbubbles or tissue and, for the present invention, fundamental frequency signals for detection of probe movement. The separator 22 may operate in a variety of ways such as by bandpass filtering the received signals in fundamental frequency and harmonic frequency bands (including super-, sub-, and/or ultra-harmonic signal bands), or by a process for fundamental frequency cancellation such as pulse inversion or amplitude modulated harmonic separation. Other pulse sequences with various amplitudes and pulse lengths may also be used for both linear signal separation and nonlinear signal enhancement. A suitable fundamental/harmonic signal separator is shown and described in international patent publication WO 2005/074805 (Bruce et al.) The separated fundamental and/or nonlinear (harmonic) signals are coupled to a signal processor 24 where they may undergo additional enhancement such as speckle removal, signal compounding, and noise elimination.

The processed signals are coupled to a B mode processor 26 and a Doppler processor 28. The B mode processor 26 employs amplitude detection for the imaging of structures in the body such as muscle, tissue, and blood cells. B mode images of structure of the body may be formed in either the harmonic mode or the fundamental mode. Tissues in the body and microbubbles both return both types of signals and the stronger harmonic returns of microbubbles enable microbubbles to be clearly segmented in an image in most applications. This characteristic is useful when forming a vascular map of the flow of microbubbles in vessels in the body. The Doppler processor 28 processes temporally distinct signals from tissue and blood flow by fast Fourier transformation (FFT) or other Doppler detection techniques for the detection of motion of substances in the image field including blood cells and microbubbles. The Doppler processor may also include a wall filter to eliminate unwanted strong signal returns from tissue in the vicinity of flow such as vessel walls. The anatomic and Doppler flow signals produced by these processors are coupled to a scan converter 32 and a volume renderer 34, which produce image data of tissue structure, flow, or a combined image of both of these characteristics. The scan converter converts echo signals with polar coordinates into image signals of the desired image format such as a sector image in Cartesian coordinates. The volume renderer 34 converts a 3D data set into a projected 3D image as viewed from a given reference point as described in U.S. Pat. No. 6,530,885 (Entrekin et al.) As described therein, when the reference point of the rendering is changed the 3D image can appear to rotate in what is known as kinetic parallax. This image manipulation is controlled by the user as indicated by the Display Control line between the user interface 38 and the volume renderer 34. Also described is the representation of a 3D volume by planar images of different image planes, a technique known as multiplanar reformatting. The volume renderer 34 can operate on image data in either rectilinear or polar coordinates as described in U.S. Pat. No. 6,723,050 (Dow et al.) The 2D or 3D images are coupled from the scan converter and volume renderer to an image processor 30 for further enhancement, buffering and temporary storage for display on an image display 40.

A graphics processor 36 is also coupled to the image processor 30 which generates graphic overlays for displaying with the ultrasound images. These graphic overlays can contain standard identifying information such as patient name, date and time of the image, imaging parameters, and the like, and can also produce a graphic overlay of a beam vector or pattern of transmit beams controllably steered by the user as described below. For this purpose the graphics processor receives input from the user interface 38. In the embodiment of FIG. 1 the graphics processor can be used to overlay a cavitation image over a corresponding anatomical B mode or flow image. The user interface is also coupled to the transmit controller 18 to control the generation of ultrasound signals from the transducer array 10 and hence the pattern of beams transmitted by the array during therapy and images produced by and effects of therapy applied by the transducer array. The transmit parameters controlled in response to user adjustment include the MI (Mechanical Index) which controls the peak intensity of the transmitted waves, which is related to cavitational effects of the ultrasound, and timing and steering of the transmitted beams for therapy and for vascular mapping as discussed below.

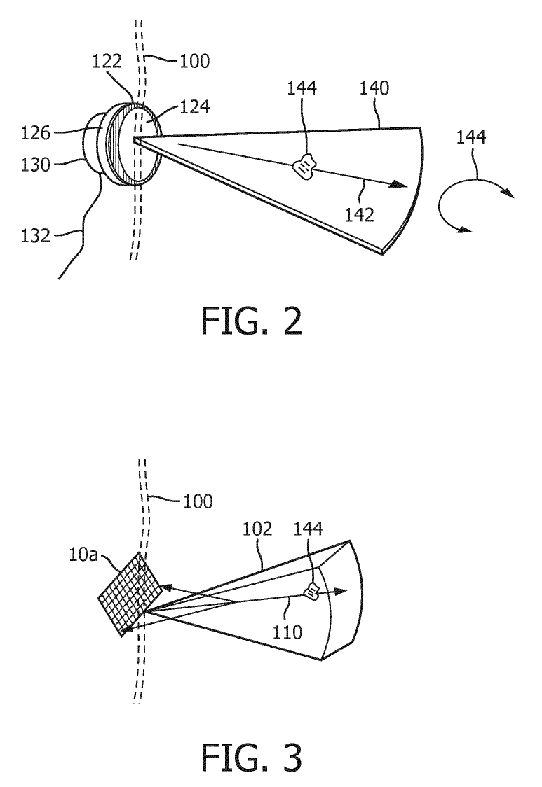

FIG. 2 illustrates the conduct of sonothrombolysis in two dimensions with a one-dimensional transducer array. In this example the transducer array 122 is a one-dimensional array which performs 2D imaging. This transducer array, like the other arrays described herein, is covered with a lens 124 which electrically insulates the patient from the transducer array and in the case of a one-dimensional array may also provide focusing in the elevation (out-of-plane) dimension. The lens is pressed against the skinline 100 for acoustic coupling to the patient. The transducer array 122 is backed with air or acoustic damping material 126 which attenuates acoustic waves emanating from the back of the array to prevent their reflection back into the transducer elements. Behind this transducer stack is a device 130 for rotating the image plane 140 of the array. The device 130 may be a simple knob or tab which may be grasped by the clinician to manually rotate the circular array transducer in its rotatable transducer mount (not shown). The device 130 may also be a motor which is energized through a conductor 132 to mechanically rotate the transducer as discussed in U.S. Pat. No. 5,181,514 (Solomon et al.) Rotating the one-dimensional array transducer 122 as indicated by arrow 154 will cause its image plane 140 to pivot around its central axis, enabling the repositioning of the image plane for full examination of the vasculature in front of the transducer array. As discussed in the '514 patent, the planes acquired during at least a 180° rotation of the array will occupy a conical volume in front of the transducer array, which may be rendered into a 3D image of that volumetric region. Other planes outside this volumetric region may be imaged by repositioning, rocking or tilting the transducer array in its headset in relation to the skull beneath the skinline 100. If a stenosis, a blood clot 144, is found in the image of the plane being imaged, the therapeutic beam vector graphic 142 can be steered by the clinician to aim and focus the beam at the stenosis 144 and therapeutic pulses applied to disrupt the microbubbles at the site of the stenosis to lyse the obstruction.

FIG. 3 illustrates a 3D imaging/therapy implementation of the present invention which uses a 2D matrix array transducer 10a. In this illustration the transducer array 10a is held against the skinline 100 of the patient with the volume 102 being imaged projected into the body. The user will see a 3D image of the volume 102 on the display of the ultrasound system in either a multiplanar or volume rendered 3D projection. The user can manipulate the kinetic parallax control to observe the volume rendered 3D image from different orientations. The user can adjust the relative opacity of the tissue and flow components of the 3D image to better visualize the vascular structure inside the brain tissue as described in U.S. Pat. No. 5,720,291 (Schwartz) or can turn off the B mode (tissue) portion of the display entirely and just visualize the flow of the vascular structure inside the 3D image volume 102.

When the site of the treatment such as a thrombus 144 is being imaged in the volume 102, a microbubble contrast agent is introduced into the patient's bloodstream. In a short time the microbubbles in the bloodstream will flow to the vasculature of the treatment site and appear in the 3D image. Therapy can then be applied by agitating or breaking microbubbles at the site of the stenosis in an effort to dissolve the blood clot. The clinician activates the “therapy” mode, and a therapy graphic 110 appears in the image field 102 on the display, depicting the vector path of a therapeutic ultrasound beam with a graphic thereon which may be aimed at a blood clot. The therapeutic ultrasound beam is manipulated by a control on the user interface 38 until the tip of the vector graphic 110 and consequently the ultrasound beam is focused at the site of the blockage. The energy produced for the therapeutic beam can be within the energy limits of diagnostic ultrasound or in excess of the ultrasound levels permitted for diagnostic ultrasound. The energy of the resulting microbubble ruptures will strongly agitate a blood clot, tending to lyse the clot and dissolve it in the bloodstream. In many instances insonification of the microbubbles at diagnostic energy levels will be sufficient to dissolve the clot. Rather than breaking microbubbles in a single rupture event, the microbubbles may be vibrated and oscillated, and the energy from such extended oscillation prior to dissolution of the microbubbles can be sufficient to lyse the clot. When vigorous activity of the microbubbles is desired to quickly lyse a blood clot or rapidly break up a large clot, it may be decided to induce cavitation at the site of the blockage to stimulate this activity. Inertial cavitation will produce the most vigorous activity, while stable cavitation will produce a lower level of microbubble agitation. The presence of cavitation at the site of the occlusion and its type is detected by a cavitation detector 50, which analyzes characteristics of r.f. echo signals to determine whether cavitation is occurring and, if so, the type of cavitation. The two different forms of cavitation produce ultrasonic backscatter of different characteristics. Stable cavitation produces a strong subharmonic response, while unstable, or inertial cavitation produces broadband noise. The cavitation detector analyzes returning echo signals, e.g., by spectral analysis, for indications of these characteristics and informs the clinician when cavitation is identified, for example by coloring the site of the therapy in an ultrasound image with a color where cavitation has been identified. If the signature for inertial cavitation is detected, for example, and stable cavitation is desired, the inertial cavitation detector 50 causes speaker 42 to issue an alarm. The user responds to this information by reducing the ultrasound output power (MI) being generated by the sonothrombolysis array. If cavitation is not detected at all, for example, by no indication of cavitation coloring of the site of the occlusion in the image, then the output power of the sonothrombolysis array is increased until cavitation is detected. Typical in situ acoustic pressures used to elicit the desired microbubble activity are generally in the range of 200 kPa to 400 kPa. This output power scaling can be accomplished automatically without user intervention via an output power control loop, for instance. The treatment is continued at the appropriate setting. Such usage allows the system to compensate for the attenuation generated by different temporal bone windows and any varying attenuation due to different acoustic properties of brain tissue.

In accordance with the principles of the present invention, a map is produced for display to the user of the vascular flow surrounding the thrombus 144 in the volumetric region 102. Any diagnostic imaging modality, such as a CT (computed tomography), CTA (computed tomography angiography), angiography, magnetic resonance imaging, or ultrasound imaging can be used to generate the vascular map in accordance with the invention.

In certain embodiments, low MI (non-destructive) ultrasound can also be used, and the echo returns monitored and/or recorded to track the vascular resonator flow over a plurality of imaging frames.

In some embodiments, ultrasound imaging can be used to generate a vascular map. The ultrasound probe used to generate the vascular map may be the same probe or different probe from the probe used for thrombolysis. In such embodiments, Doppler processing may be used. The Doppler processing can comprise power Doppler processing, in which the magnitudes of the flow signals at points inside the volume are estimated and displayed in a volume rendering in colors depicting the magnitudes of the flow signals. A 3D image is normally displayed in overlay with a B mode image of the vessel tissue so that the flow is shown inside the vessels carrying the flow. But in a 3D rendering the tissue will obscure much of the flow behind the outer surface of the volume, and so a preferred display technique is to display the flow alone, so that only the paths of the microbubble and blood flow are displayed, as described in U.S. Pat. No. 5,474,073 (Schwartz et al.) In a preferred implementation of the present invention, the Doppler processing used is colorflow Doppler, in which flow signals above a noise threshold are displayed in colors depicting the direction of flow at each point in a vessel, and color shading depicting the flow velocity. The resulting rendered 3D image, again displayed without the usual B mode tissue overlay, is a map appearing as a 3D web of flow paths of the cranial vasculature, with colors indicating the velocity and direction of flow in the vessels. The production of such a colorflow Doppler vascular map is described and illustrated in U.S. Pat. No. 6,682,483 (Abend et al.), for example. Such a 3D map of the flow around a thrombus will indicate the location of microbubbles in the vessel where the thrombus is lodged and, importantly for an implementation of the present invention, the flow and speed of flow of microbubbles toward thrombus, that is, the flow paths which are supplying fresh microbubbles to the therapy site.

FIG. 4 illustrates a headset 62 for a sonothrombolysis array probe 12 of the present invention mounted on the head 60 of a mannequin. The sides of the head of most patients advantageously provide suitable acoustic windows for transcranial ultrasound at the temporal bones around and in front of the ears on either side of the head. In order to transmit and receive echoes through these acoustic windows the transducer arrays must be in good acoustic contact at these locations which may be done by holding the transducer arrays against the head with the headset 62. An implementation of the present invention may have a snap-on deformable acoustic standoff which allows the transducer array to be manipulated by its conformal contact surface and aimed at the blood clot and surrounding blood vessels within the brain while maintaining acoustic contact against the temporal window. An array 10 is integrated into the probe housing 12 which allows it to address the requirements of stable positioning and tight coupling to the patient's temporal bone. The illustrated probe housing is curved by bending the probe handle by 90°, which makes the probe more stable when attached to the headset 62. The acoustic coupling objective is facilitated by integrating a mating spherical surface into the probe handle, which allows it to pivot in the headset 62 until it is strongly and tightly coupled to the temporal window of the patient.

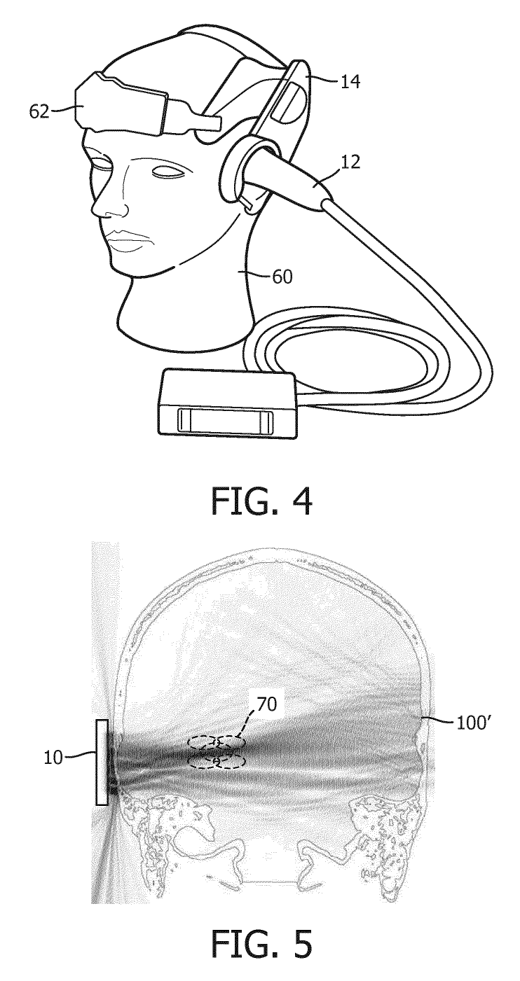

FIG. 5 is an illustration of a patient's head 100′ with a transducer array 10 at the left side of the head transmitting therapeutic ultrasound beams focused at spots 70 around a thrombus in the brain. As the multiple spots 70 show, the sonothrombolysis treatment does not consist of a single target location at the thrombus, but a plurality of target locations around the thrombus, which cause lysis all around the periphery of the clot. The gray areas in the drawing illustrate the presence of ultrasound energy in the head, which is seen to come to a focus in the darker areas at the site of the thrombus. But bands of lighter gray are seen above and below the target sites 70, which are energy produced by grating lobes, side lobes of the main beam lobe, which also contain ultrasound energy. Grating lobes are most acute when the beam is steered off-axis, that is, at an angle which is not normal to the plane of the array 10. These grating lobes can undesirably disrupt and dissolve microbubbles away from the main beam focus, microbubbles which desirably should be left unaffected in vessels that are supplying fresh microbubbles to the therapy site 70. Since the presence of these grating lobes are a known acoustic phenomenon, their presence should be taken into account when planning the sonothrombolysis therapy.

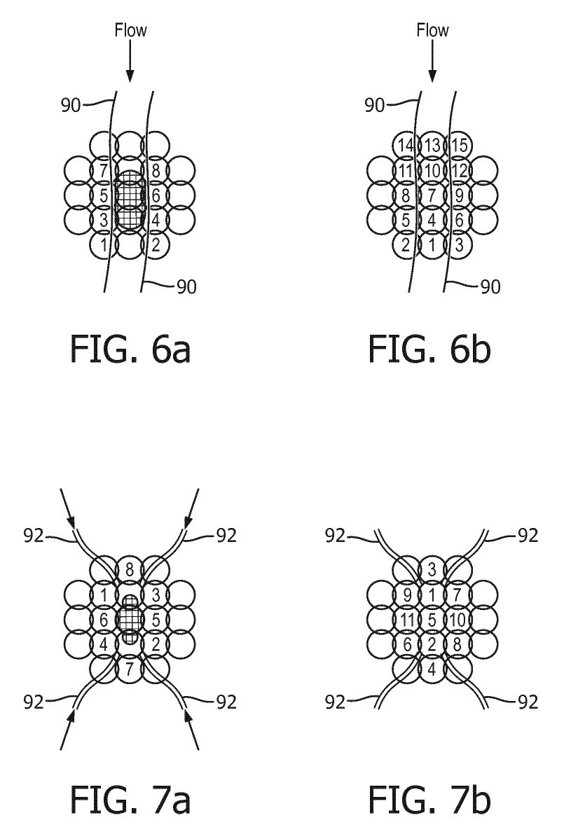

In accordance with the principles of the present invention, the flow characteristics which conduct fresh microbubbles to the site of the thrombus, such as the speed and direction of the flow, information which is present in the vascular map of the blood and microbubble flow, are used to plan the pattern of therapy beams used to lyse a thrombus. FIGS. 6a and 6b are illustrations of the use of knowledge of the direction of flow to plan a therapeutic beam pattern. In FIG. 6a, a blood clot 144 is lodged in a blood vessel 90 with flow occurring from the top of the vessel to the thrombus in the image. Overlaying the blood vessel in the drawing is a pattern of twenty-one circles depicting the focal regions of twenty-one possible therapeutic beams aimed at and around the thrombus 144 and blood vessel 90. The numbers in the circles indicate the sequence in which the therapeutic beams are transmitted in this example. Since the flow of blood containing microbubbles is from top to bottom in the illustration as indicated by the arrow, fresh microbubbles are arriving at the site of the thrombus from the top. Thus, it is undesirable to transmit the therapeutic beam sequence starting at the top, as this would disrupt or destroy microbubbles that are en route to the treatment site, where they are more beneficially disrupted or destroyed to lyse the clot. Accordingly, the illustrated plan of therapy for this clot and blood vessel starts below the clot with the sequential transmission of therapy beams 1 and 2, then proceeding upward (upstream) with therapy beams 3 and 4, then therapy beams 5 and 6, then therapy beams 7 and 8. This sequence of therapy beams allows a flow of fresh microbubbles to the clot and beam targets to continue unimpeded until the very end of the beam sequence. After the sequence has been executed, therapy is paused to allow fresh microbubbles to reinfuse the therapy site and flow through the vessel to the obstruction. The length of this pause is dependent on the flow rate of the microbubbles, a rate which is gleaned from the velocities found in the colorflow Doppler flow map, and may last for several seconds, for instance. When the therapy site has been reinfused with fresh microbubbles, the beam sequence is executed again. This sequence of beam transmission followed by microbubble replenishment continues until the clot is fully lysed or conditions change which leads to a modification of the plan. For example, after the clot has been partially broken up, microbubbles may flow around the clot remnants at a greater rate, which causes microbubble replenishment to occur more rapidly and calls for a shortening of the pause between therapy beam sequences.

The therapy beam sequence of FIG. 6a is premised on the assumption that the transmission of beams at either side of the blood vessel 90, e.g., therapy beams 1 and 2, are sufficient to also disrupt microbubbles in the blood vessel between the adjacent beams. If this is not the case, the eight-beam therapy beam pattern of FIG. 6a may be modified to produce the therapy beam sequence of FIG. 6b. In this beam pattern, the first therapy beam of each row is directed to the vessel 90, with the adjacent beams following to the left and the right of the vessel. This results in a fifteen-beam therapy beam pattern as shown in this example, which thoroughly insonifies the lumen and sides of vessel 90, again starting most distantly (downstream) from the microbubble supply and continuing upwards toward it.

FIGS. 7a and 7b illustrate therapy plans which have the objective of spatially separating successive therapy beam spatially, so that the transmission of one beam at one target site will have the least disruptive effect on microbubbles at the next target site to be insonified. In these examples the site of the thrombus 144 is in a capillary bed which is receiving flows of microbubbles from multiple blood vessels 92 which flow in different directions, so there is no single flow direction which dictates a flow-direction-based sequence. In FIG. 7a the therapy beam pattern begins with beam 1 transmitted to the upper left of the clot 144, followed by transmission of the next beam 2 to the lower right of the clot. The sequence continues with beam 3 transmitted to the upper right of the clot, followed by beam 4 transmitted to the lower left. Next, therapy beam 5 is aimed to the right of the clot, followed by beam 6 aimed to the left. The sequence concludes with therapy beam 7 aimed below the clot and therapy beam 8 aimed above it. It is seen that this sequence is designed to spatially separate successive therapy beams to minimize microbubble disruption at an immediately following target site.

FIG. 7a assumes that the therapy beams transmitted around the thrombus will have sufficient microbubble-disrupting overlap so that transmission at the center of the pattern is unnecessary. FIG. 7b shows another therapy beam pattern which thoroughly covers both the thrombus 144 and its periphery. (The thrombus 144 is omitted from this drawing for clarity of illustration of the beam pattern.) This sequence begins with therapy beam 1 aimed at the top of the thrombus, followed by therapy beam 2 aimed at the bottom. Then, beam 3 is aimed above the clot and beam 4 below it. Beam 5 follows at the center of the thrombus. The sequence concludes with six beams alternating from side-to-side and from top to bottom of the thrombus. This sequence reduces disruption at a successive target site from a beam transmitted to a previous target site, and allows time for some reinfusion of microbubbles in a targeted region before the next therapy beam is directed to that region. As before, after completion of these sequences, a pause in transmission occurs to allow the replenishment of fresh microbubbles to the site of the thrombus. Since vessels 92 are seen to be smaller than vessel 90 and are feeding the supply of microbubbles to a capillary bed where thrombus 144 is lodged, microbubble replenishment will generally take longer, calling for a longer pause between therapy sequences, than was the case in the example of the large vessel 90 in FIGS. 6a and 6b.

Other factors revealed by the vascular flow map may also be taken into consideration when planning the sonothrombolysis therapy. For instance, the presence of grating lobes that undesirably disrupt microbubbles at target sites to which therapy beams have not yet been directed in a sequence can also be considered, as explained in conjunction with FIG. 5. The therapy beam sequence can be arranged to allow time for replenishment of microbubbles at a target site disrupted by grating lobe energy from a preceding beam before a therapy beam is subsequently aimed at that target site. Potential beam directions which would be directed to a region of the therapy site which cannot be infused with microbubbles, such as the region of a vessel downstream from a fully occluding clot, can be omitted from the plan so that treatment can be directed to locations with microbubble infusion where treatment would be more effective. And as mentioned above, the treatment plan should be re-evaluated during therapy as clot lysis opens clotted vessels and changes the vascular flow dynamics at the treatment site, in case a more effective plan becomes viable.

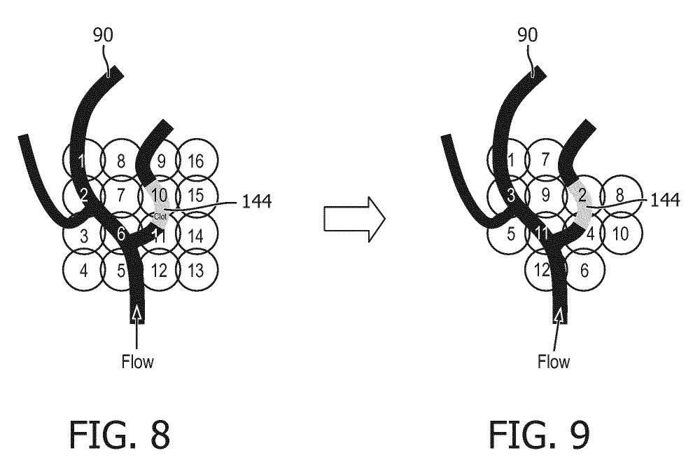

The starting point for development of a treatment plan is generally a default treatment plan which has been predetermined and stored in memory in the ultrasound system. A default treatment plan is one which is composed of a large number of individual treatment sites such as that shown in FIG. 8, which is generally influenced by the size of the focal zone of the therapy ultrasound array 10. The default therapy beam pattern is frequently arranged in a rectangular or circular pattern, and fully covers (or “paints”) a typical target volume which contains a clot and surrounding tissue margin. The treatment plan is traversed a multitude of times during sonothrombolysis treatment, so as to deliver the required ultrasonic stimulation and microbubble dose to the clot target in order to achieve clot lysis. In the example of FIG. 8, a sixteen-beam default beam sequence is shown which steers the succession of therapy beams alternately from top to bottom and bottom to top, and left to right. This therapy beam pattern is shown in registration with the flow of branches of a blood vessel 90, one branch of which is occluded by a thrombus 144. The therapy beam pattern of the default treatment plan is stored in system memory and it or a modified treatment plan is executed by the transmit controller 18 which controls the transmission timing, steering and focusing of therapy beams by the array 10, with the focal depth and location set in response to the setting of the therapeutic beam vector graphic 142, 110 by a clinician.

Starting from this default treatment plan and its beam pattern, the microbubble flow direction and vessel topography revealed by the vascular flow map indicate that a more effective treatment plan can be developed by considering these factors. One such treatment plan beam sequence is shown in FIG. 9. This treatment plan combines a number of the planning considerations detailed above. In consideration of the direction of flow, the therapy beam sequence starts by transmitting the first beam 1 to the top of vessel 90, a site most distant downstream from the flow of replenishing microbubbles at the bottom of the vessel. The next beam 2 is spatially separated to the right from the first beam, aimed at the clot 144. Then beam 3 is aimed back at the left branch of the vessel before beam 4 returns to the site of the clot in the right branch of the vessel. The sequence continues laterally back and forth and toward the source of fresh microbubbles at the bottom of the vessel. Then the remainder of the treatment plan continues with beam 7 back at the top of the blood vessel, with subsequent beams alternating back and forth until again reaching the bottom of the vessel with beam 12. The other four possible therapy beam sites are omitted from the plan, as they are spatially offset from the vessel 90 and thus unlikely to have a significant therapeutic effect.

In a constructed implementation of the present invention, the formulation of the treatment plan and its therapeutic beam sequencing can be done manually by a clinician, or automatically by a therapy planning program or module of the ultrasound system which is programmed to do so. For instance, the clinician can aim the therapeutic beam vector 142, 110 at the thrombus to set the depth and location of the beam pattern transmitted under control of the transmit controller 18. Then the clinician can call up an image of the default treatment plan and, by observing flow characteristics in the vascular map such as the direction of microbubble flow to the thrombus, set the sequence in which selected beams are to be transmitted as illustrated in FIGS. 6, 7, and 9, and the duration of the pause interval between therapy beam transmission from the flow velocity observed in the map. In a more automated implementation, a treatment program would respond to the setting of the therapeutic beam vector 142,110 by setting up the transmit controller 18 for therapeutic beam transmission in the indicated direction and focused at the indicated depth. The treatment program would then analyze the vascular flow map for paths of microbubble flow toward the thrombus and the rate of flow, and set the therapeutic beam sequencing and interval between times of beam pattern transmission for the transmit controller 18. Therapy would then commence, with periodic checks of the vascular map to see if any change in the treatment plan is warranted.

It should be noted that an ultrasound system suitable for use in an implementation of the present invention, and in particular the component structure of the ultrasound system described in FIG. 1, may be implemented in hardware, software or a combination thereof. The various embodiments and/or components of an ultrasound system, for example, the modules, or components and controllers therein, also may be implemented as part of one or more computers or processors. The computer or processor may include a microprocessor. The microprocessor may be connected to a communication bus, for example, to access a PACS system or a data network. The computer or processor may also include a memory. The memory devices may include Random Access Memory (RAM) and Read Only Memory (ROM) or other digital or analog signal storage components. The computer or processor further may include a storage device, which may be a hard disk drive or a removable storage drive such as a floppy disk drive, optical disk drive, solid-state thumb drive, and the like. The storage device may also be other similar means for loading computer programs or other instructions into the computer or processor.

As used herein, the term “computer” or “module” or “processor” or “workstation” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of these terms.

The computer or processor executes a set of instructions that are stored in one or more storage elements, in order to process input data. The storage elements may also store data or other information as desired or needed. The storage element may be in the form of an information source or a physical memory element within a processing machine.

The set of instructions of an ultrasound system including those controlling the acquisition, processing, and transmission of ultrasound images as described above may include various commands that instruct a computer or processor as a processing machine to perform specific operations such as the methods and processes of the various embodiments of the invention. The set of instructions may be in the form of a software program. For instance, the ultrasound system of FIG. 1 may be programmed with instructions executing an algorithm which applies the treatment planning considerations enumerated above to a vascular flow map and an indicated thrombus location to develop an effective sequence of therapy beam sequencing. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine. In the ultrasound system shown in FIG. 1, for instance, software instructions are conventionally employed to create and control the display of a vascular map and user control functions described above, and analysis such as application of the treatment planning considerations.

Furthermore, the limitations of the following claims are not written in means-plus-function format and are not intended to be interpreted based on 35 U.S.C. 112, sixth paragraph, unless and until such claim limitations expressly use the phrase “means for” followed by a statement of function devoid of further structure.

1. An ultrasound system adapted to perform therapy on a subject comprising:
an array of transducer elements, configured to transmit a plurality of therapeutic ultrasonic energy beams aimed at a therapeutic site;
a diagnostic imaging modality which is adapted to produce a vascular map of the therapeutic site, the vascular map comprising a volume rendering of flow signals acquired at the therapeutic site;
a therapy beam transmit controller, responsive to a treatment plan formulated in consideration of the flow signals, which is adapted to transmit a variable sequence of differently steered therapy beams to the therapeutic site during a plurality of transmission intervals.

2. The ultrasound system of claim 1, wherein the treatment plan is further formulated to determine an order in which the differently steered therapy beams are transmitted.

3. The ultrasound system of claim 2, wherein the treatment plan is further formulated to determine a number of differently steered therapy beams which are transmitted.

4. The ultrasound system of claim 3, wherein the treatment plan is further formulated to determine a length of a pause between successive transmission intervals.

5. The ultrasound system of claim 4 wherein the length of the pause between successive transmission intervals is determined in consideration of a flow velocity identified by the vascular map.

6. The ultrasound system of claim 2, wherein the treatment plan is further formulated to determine the order in which the differently steered therapy beams are transmitted in consideration of a flow direction identified by the vascular map.

7. The ultrasound system of claim 6, wherein the treatment plan is further formulated to determine the order in which the differently steered therapy beams so that a later-transmitted beam in the sequence is upstream in relation to the flow direction from an earlier-transmitted beam.

8. The ultrasound system of claim 2, wherein the treatment plan is further formulated to determine the order in which the differently steered therapy beams are transmitted in consideration of a spacing between successively transmitted therapy beams.

9. The ultrasound system of claim 2, wherein the treatment plan is further formulated to determine the order in which the differently steered therapy beams are transmitted in consideration of grating lobes of the steered therapy beams.

10. The ultrasound system of claim 1, wherein the array of transducer elements is further adapted to receive ultrasonic echo signals;
wherein the diagnostic imaging modality further comprises the ultrasound system performing the therapy, and further comprising:
a user control adapted to control an ultrasound image graphic, wherein the vascular map further comprises a therapeutic beam vector graphic adapted to indicate a location of a therapy site in an ultrasound image in response to the user control.

11. The ultrasound system of claim 10, further comprising a treatment program which is adapted to determine a therapy beam steering direction and a focal depth in response to the user control of the therapeutic beam vector graphic.

12. The ultrasound system of claim 11, wherein the treatment program is further adapted to determine a sequence of therapy beam transmission in response to the vascular map.

13. The ultrasound system of claim 11, wherein the treatment program is further adapted to determine a pause between sequences of therapy beam transmission in response to the vascular map.

14. The ultrasound system of claim 11, wherein the treatment program is further adapted to determine a number of therapy beams transmitted in a sequence in response to the vascular map.

15. The ultrasound system of claim 11, wherein the treatment program is further adapted to determine a spacing between successively transmitted therapy beams in response to the vascular map.

16. The ultrasound system of claim 1, wherein the diagnostic imaging modality is selected from the group consisting of computed tomography, computed tomography angiography, angiography, magnetic resonance imaging, and ultrasound imaging.
